(19) United States
(12) Patent Application Publication
(10) Pub. No.: US 2026/0020881 A1
(43) Pub. Date: Jan. 22, 2026

(54) Patient-Specific Pin Registration Guide and Method for Properly Locating Rigid External Halo Devices

(71) Applicant: KLS Martin, L.P., Jacksonville, FL (US)

(72) Inventor: Brett Walter, Jacksonville, FL (US)

(73) Assignee: KLS Martin, L.P., Jacksonville, FL (US)

(21) Appl. No.: 19/274,439

(22) Filed: Jul. 18, 2025

Related U.S. Application Data
(60) Provisional application No. 63/673,136, filed on Jul. 18, 2024.

(51) Int. Cl. A61B 17/64; A61B 17/00; G06F 30/12

(52) U.S. Cl. CPC A61B 17/6433; G06F 30/12; A61B 2017/00526

(57) ABSTRACT

A patient-specific pin registration guide for properly orienting a halo on head of patient and the method of its creation and use wherein the pin registration guide has an upper portion with pin locator notches and a lower conforming portion that matches the anatomical configuration of the patient's ear, such that with the lower conforming portion mounted onto the ear of the patient, the pin locator notches will be precisely located for proper positioning of a halo device.

### BACKGROUND OF THE INVENTION

This invention relates to rigid external fixation devices utilized in cranio-maxillary-facial (CMF) surgery or treatment. These devices are often referred to as halos, and are circular, oval or U-shaped devices affixed around or onto a patient's head using laterally mounted pins. The halos may be used for example to immobilize the head of the patient or to provide a fixed base for orthodontic or CMF distraction or surgery.

Because the halo is mounted over soft tissue, proper location or registration of the halo onto the patient's head is difficult, as there are no reference points to guide the surgeon a to proper placement of the pins.

It is an object of this invention to provide a novel method and device for properly locating the halo onto the patient's head, wherein a pin registration guide is created to indicate the locations for the pins, the pin registration guide conforming or mating with an external anatomical feature of the patient's head, such as an ear or the nose. The pin registration guide is created using scanning software to produce a virtual model of the skull and the soft tissue external anatomical features, whereby the pin registration guide is designed to conform and properly mount onto one or more external anatomical features in a single, desired orientation. Thus, the pin registration guide is patient-specific.

### SUMMARY OF THE INVENTION

The invention in various embodiments comprises a customized, patient-specific, halo pin registration guide which indicates to the surgeon the proper location on the patient's head for placement of the halo fixation pins, thereby ensuring that the halo is properly located. The pin registration guide comprises a plurality of pin locator notches, preferably semi-circular in configuration and positioned on the upper portion of the pin registration guide. The lower portion of the pin registration guide defines a registration portion, the registration portion being designed to conform or mount onto at least a portion of an external anatomical feature or prominence of the head, such as an ear or nose, whereby the registration portion and therefore the pin registration guide itself will only mount onto the external anatomical feature in a single, desired orientation, thereby ensuring proper location of the pins. For example, for a pin registration guide conforming to an ear, the lower portion comprises an upper curved segment configured to rest on the tissue above the cartilage between the helix of the ear and the temporal skin, as well as a lower base segment configured to reside within and fully mate with the concha surrounding the ear canal of the ear.

The invention is also the method of designing and utilizing the pin registration guide such that it is specific to an individual patient. A virtual 3-D model of the patient's cranium and the external soft tissue is created by CT-scan or similar imaging equipment. The creation of virtual 3-D images for surgical planning and the creation of implants based on the virtual 3-D images is well known in the art. Based on input from the surgeon, the desired locations on the head of the patient for the halo fixation pins are determined on the virtual model. A technician then designs a virtual pin registration guide comprising an upper portion with pin location notches corresponding to the desired pin locations for the halo and a lower conforming portion of the pin registration guide conforming to the ear's external anatomical features, namely the concha and the arch at the base of the helix. Based on this virtual design, a physical pin registration guide is then manufactured. With the pin registration guide properly oriented onto the patient's ear the surgeon can either mark the pin locations or, most preferably, position the halo onto the patient's head such that the fixation pins correspond to the pin location notches. The fixation pins are then tightened to secure the halo on the patient's head. The pin registration guide is then removed by sliding it off the ear and away from the pins. Alternatively, the pin registration guide can be cut for easy removal.

### BRIEF DESCRIPTION OF THE DRAWINGS

FIG. 1 is a side view of an embodiment of the pin registration guide configured for use with a halo having a linear array of fixation pins, shown positioned on a virtual 3-D model of a human head.

FIG. 2 is a perspective view of the embodiment of FIG. 1.

FIG. 3 is a rear view of the embodiment of FIG. 1.

FIG. 4 is a side view of an alternate embodiment of the pin registration guide configured for use with a halo having a linear array of fixation pins, shown positioned on a virtual 3-D model of a human head.

FIG. 5 is a side view of the embodiment of FIG. 4 and a halo properly positioned in alignment with the pin registration guide, prior to insertion of the halo fixation pins.

FIG. 6 is a side view of an alternate embodiment of the pin registration guide configured for use with a halo having a non-linear array of fixation pin apertures, shown positioned on a virtual 3-D model of a human head.

FIG. 7 is a side view of the mounting plate portion of a halo having a non-linear array of fixation pin apertures matching the non-linear array of pin registration notches on the pin registration guide of FIG. 6.

FIG. 8 is a side view showing the upper portion of the pin registration guide of FIG. 6, the mounting plate portion of the halo of FIG. 7 with the fixation pin apertures of the halo properly aligned with pin registration notches of the pin registration guide, also showing cylindrical representations of the halo fixation pins inserted through the halo mounting plate prior to removal of the pin registration guide.

### DETAILED DESCRIPTION OF THE INVENTION

With reference to the drawings, the invention will be described in detail with regard to the pin registration guide and the method of creation and use of the pin registration guide. It is to be understood that the drawings are for illustrative, enabling and definitional purposes, and are not meant to limit the scope of the invention. The terms upper or superior refer to the direction toward the top of the head and the terms lower and inferior refer to the opposite direction toward the bottom of the head. The term patient-specific refers to a customized pin registration guide with portions conforming to the anatomy of the particular patient.

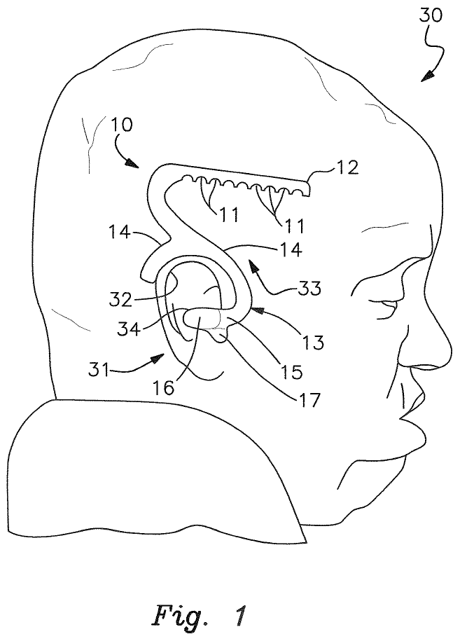

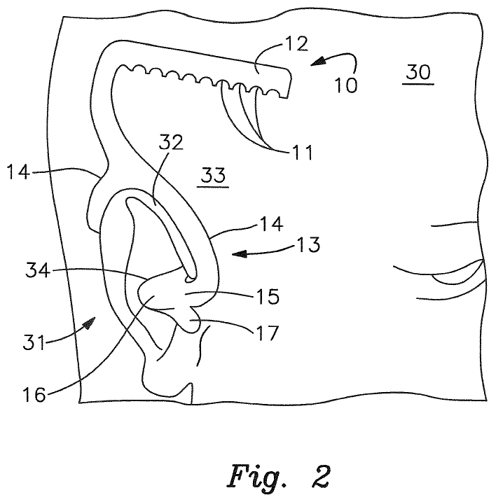

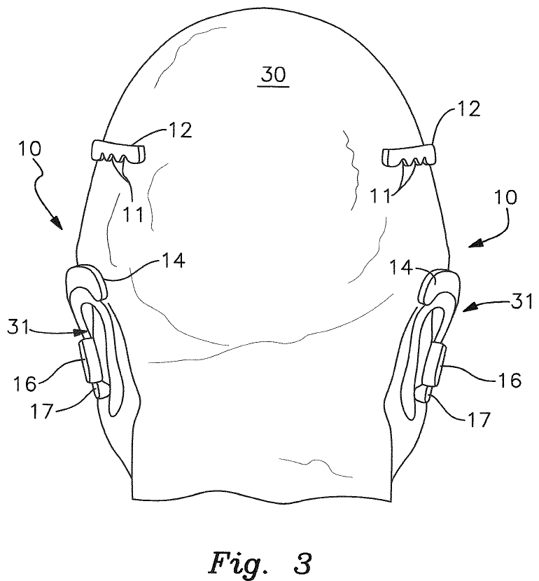

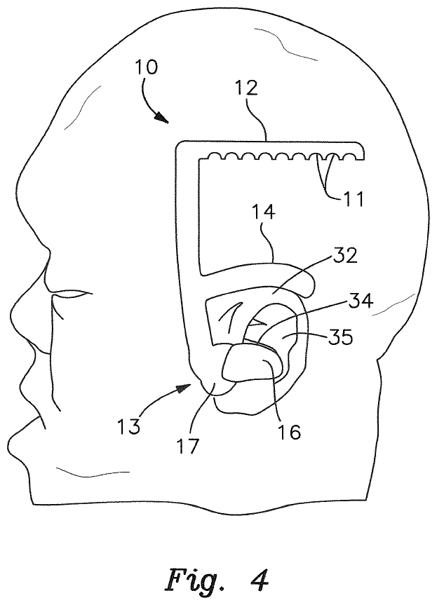

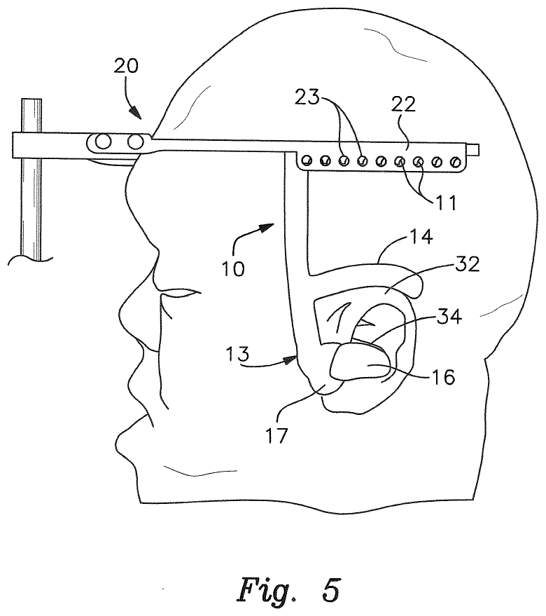

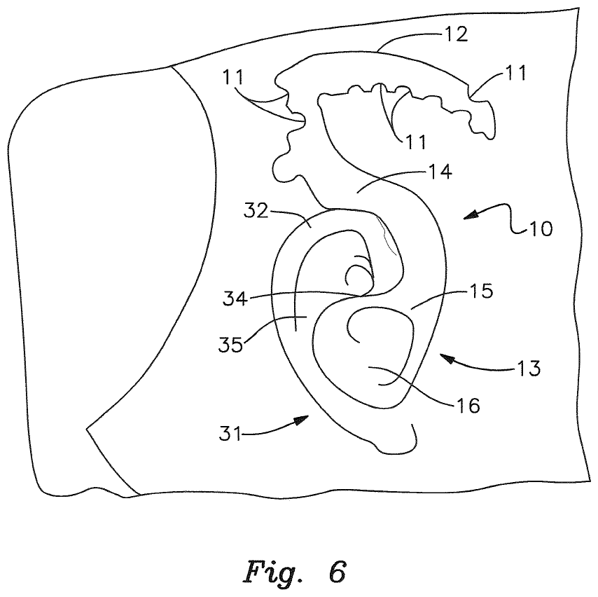

FIGS. 1 through 3 illustrate an embodiment of the pin registration guide 10 wherein the upper portion 12 comprises a straight or linear array of pin locator notches 11. FIGS. 4 and 5 illustrate an alternate embodiment of the pin registration guide 10 similar to that of FIGS. 1 through 3, but with the lower conforming portion 13 having a different configuration. FIGS. 6 through 8 illustrate an alternate embodiment of the pin registration guide 10 wherein the upper portion 12 comprises a non-linear array of pin locator notches 11.

To produce the pin registration guide 10, a patient's head 30 is imaged in known manner using a CT scanner or similar imaging equipment to produce a virtual 3-D model which includes the external skin and external anatomical features or prominences, such as an ear or the nose. The surgeon is then consulted to determine the desired location of the contact points for the fixation pins 21 of the halo 20. The location of the contact points for the fixation pins 21 will be determined based on various factors, such as malformation of the cranium, weak cranial bone segments, damaged or missing cranial bone segments, etc.

Once the contact points are determined, the technician designs a virtual pin registration guide 10 conforming to the soft tissue 3-D configuration. The upper portion 12 of the pin registration guide 10 is provided with an array of pin locator indents or notches 11 corresponding to the predetermined contact points for the halo fixation pins 21. Most preferably, the pin locator notches 11 are semicircular or otherwise not fully bounded such that the upper portion 12 may be slid off of the halo fixation pins 21 once the halo fixation pins 21 have been inserted through the pin apertures 23 of the mounting plate portion 22 of the halo 20 to secure the halo 20 to the patient's head 30. Alternatively, circular bounded apertures (not shown) may be utilized if the pin registration guide is used by the surgeon only for marking the location of the contact points for the halo fixation pins 21.

The technician then designs the lower conforming portion 13 of the virtual pin registration guide 10, which is joined to the upper portion 12, to conform or mate with the patient's ear 31, the conformation being such that the pin registration member 10 can only be mounted in one desired orientation relative to the ear 31, thus ensuring that the upper portion 12 is properly located on the patient. The lower conforming portion 13 comprises two segments—an upper curved segment 14 which resides along the junction between the base of the ear or helix 32 and the temporal skin 33 of the patient, and a lower base segment 15 shaped to fit into and mate with the concha 34, the hollow central part and largest cavity of the external ear 31, such that the irregular configuration of the inner surface of the lower base segment 15 matches the irregular outer surface of the concha 34. The convex lower base segment 15, unlike Bluetooth earbuds for example which have a standard shape for use in all ears, fully mates with the convex concha 34 such that, in combination with the upper curved segment 14, the lower conforming portion 13 will not rotate, shift or otherwise move from the single desired orientation once mounted onto the patient.

This process is repeated for the other ear 31 of the patient, such that a virtual pin registration guide 10 is designed for both the right ear and the left ear.

The physical pin registration guides 10 are then produced from the design specs and parameters determined from the virtual 3-D model. The surgeon then places the pin registration guides 10 onto the patient by positioning the helix-adjacent upper curved segment 14 between the ear 30 and the temporal skin 33 and mating the lower conforming segment 15 of the pin registration guide 10 within the concha 34. In this manner, the pin locator notches 11 of the upper portion 12 reside in the desired locations for the contact points of the halo 20 on both sides of the patient's head 30.

The surgeon then positions the halo 20 onto the patient's head 30 such that the pin apertures 23 located on the halo mounting plate portions 22 are aligned with the pin locator notches 11. The surgeon then inserts the fixation pins 21 through the pin apertures 23 to secure the halo 20 to the patient's head 30. The pin registration guides 10 are then removed by separating the lower conforming portion 13 from the concha 34 and behind the helix 32 and sliding the upper portion 12 off of the halo fixation pins 21. Alternatively, the upper portion 12 and lower conforming portion 13 of the pin registration guide may be severed from each other and separately removed. As previously mentioned, it is also possible to first properly position the pin registration guide 10 on the patient and then use the pin locator notches 11 as marking guides, with the pin registration guide 10 being removed prior to installation of the halo 30.

An embodiment of a pin registration guide 10 is illustrated in FIGS. 1 through 3. In this embodiment the pin registration guide 10 comprises an upper portion 12 having a straight or linear array of pin locator notches 11. The upper portion 12 is attached to the lower conforming portion 13. The upper curved segment 14 of the lower conforming portion 13 extends forward and rearward between the helix 32 and temporal skin 33 and rests on the junction where the ear joins the head. At the forward end the upper curved segment 14 transitions into lower base segment 15. The inner surface of the convex lower base segment 15 is sized and configured to match the size and configuration of the concave outer surface of the concha 34, the deep recess or cavity forming the hollow central part of the ear 31 surrounding the ear canal. In this manner the lower base segment 15 is irregularly shaped with a thick, somewhat elongated, main body portion 16 having a forward extending short leg 17, and mates with the irregularly shaped concha in a snug manner. The inner surface of the pin registration guide 10 conforms to the shape and configuration of the patient's head, such that it abuts the tissue surface of the patient.

FIGS. 4 and 5 illustrate an alternate embodiment of the pin registration guide 10 similar to that of FIGS. 1 through 3, but with a lower conforming portion 13 of a different configuration. In this embodiment the upper curved segment 14 is straighter toward the rear and does not conform to the front of the ear. FIG. 5 shows the pin apertures 23 of the mounting plate portion 22 of the halo 20 aligned with the pin locator notches 11 of the pin registration guide 10, such that the halo 10 will be in the proper position and orientation when the pins are inserted and the halo is secured onto the patient's head 30.

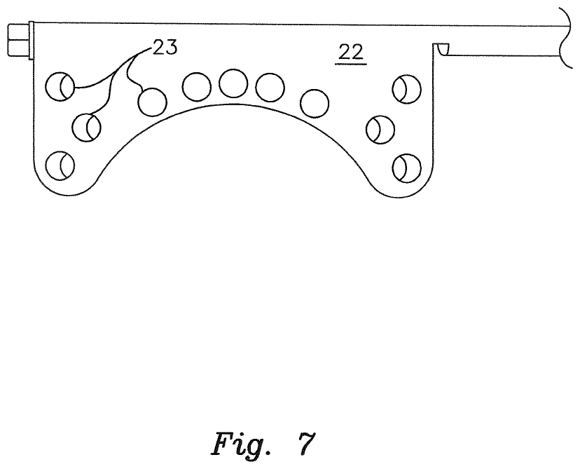

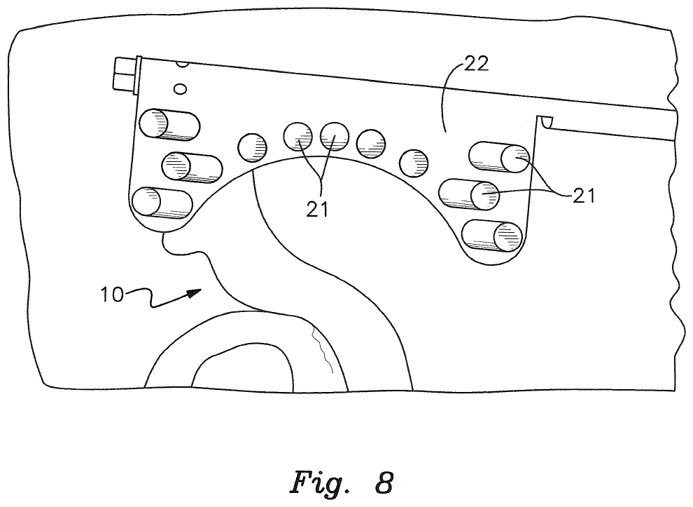

FIGS. 6 through 8 illustrate an alternate embodiment of the pin registration guide 10 wherein the upper portion 12 comprises a non-linear array of pin locator notches 11. In this embodiment the pin registration guide 10 is used with a halo 10 having a curved mounting plate portion 22 as shown in FIGS. 7 and 8. The non-linear array of pin locator notches 11 matches the non-linear array of pin apertures 23 on the halo 10, which in this case comprise one set of pin apertures 23 in a curved alignment and several pin apertures 23 in forward and rearward positions. The fixation pins 21 are rendered as cylinders in FIG. 8. In this embodiment the main body portion 16 is expanded to cover a greater portion of the concha 34 as well as part of the antihelix 35.

It is understood that equivalents and substitutions for elements set forth above may be obvious to those of skill in the art. Therefore, the true scope and definition of the invention is to be as set forth in the following claims.

I claim:

1. A patient-specific, halo pin registration guide configured to mount onto a human head, the pin registration guide comprising an upper portion and a lower conforming portion;
the upper portion comprising a plurality of pin locator notches;
the lower conforming portion of the pin registration guide defining a registration portion, the registration portion designed to conform and mount onto a human ear, wherein the registration portion will only mount onto the ear in a single, desired orientation, thereby ensuring proper location of the pins.

2. The pin registration guide of claim 1, wherein the lower portion comprises an upper curved segment configured to rest on the tissue above the cartilage between the helix of the ear and the temporal skin; and
wherein the lower base segment is configured to reside within and fully mate with the concha of the ear.

3. The pin registration guide of claim 2, wherein the pin locator notches are semi-circular in configuration.

4. A method of manufacturing a patient-specific, halo pin registration guide configured to mount onto a human head comprising the steps of:
creating a virtual 3-D model of a patient's cranium and the external soft tissue of the patient's ear by imaging equipment;
determining the desired locations on the virtual 3-D model for halo fixation pins;
designing a virtual pin registration guide comprising an upper portion with pin location notches corresponding to the desired halo pin locations and a lower conforming portion of the pin registration guide conforming to the ear's external anatomical features; and
creating a physical pin registration guide based on the specs of the virtual pin registration guide.

5. The method of claim 4, wherein the step of creating a virtual 3-D model comprises including the concha and the arch at the base of the helix of the ear; and
wherein the step of designing the virtual pin registration guide comprises designing the lower conforming portion with an upper curved segment configured to rest on the arch at the base of the helix of the ear and a lower base segment configured to reside within and fully mate with the concha of the ear.
